(19) United States
(12) Patent Application Publication
(10) Pub. No.: US 2008/0092249 A1
(43) Pub. Date: Apr. 17, 2008

(54) Method of nuclear transfer

(75) Inventors: Ian Lewis, Victoria (AU); Gabor Vajta, Tjele (DK); Tayfur Tecirlioglu, Burwood (AU)

Correspondence Address: BROWDY AND NEIMARK, P.L.L.C.;624 NINTH STREET, NW, SUITE 300, WASHINGTON, DC, 20001-5303, US

(73) Assignee: MONASH UNIVERSITY, Victoria (AU)

(21) Appl. No.: 11/808,383

(22) Filed: Jun. 8, 2007

Related U.S. Application Data
(63) Continuation of application No. 10/475,168, filed on Apr. 21, 2004, now abandoned.

(30) Foreign Application Priority Data
Apr. 20, 2001 (AU) PR 4514

(51) Int. Cl. A01K 67/00; C12N 15/00

(52) U.S. Cl. 800/8; 800/14; 800/16; 800/21

(57) ABSTRACT

The present invention relates to nuclear methods and embryos developed therefrom. In particular, the present invention relates to a method of nuclear comprising the step of transferring a somatic cell nuclei into a zona pellucida-free, enucleated oocyte.

The present application is a Continuation of application Ser. No. 10/475,168, filed Apr. 21, 2004.

### FIELD OF THE INVENTION

The present invention relates to nuclear transfer methods and embryos developed therefrom. Methods of culturing embryos and reconstituting animals from the embryos generated by the nuclear transfer methods of the present invention are also included.

### BACKGROUND OF THE INVENTION

The potential benefits of nuclear transfer have been reviewed recently in a number of publications (Galli et al., 1999; Colman, 1999; Wells & Powell, 2000; Lewis et al., 2001; Trounson, 2001). Methods for nuclear transfer have been sought and developed in earnest over the past two decades and are described in many references (See, for example, Campbell et al., Theriogenology, 43: 181 (1995); Collas et al., Mol. Report. Dev., 38: 264-267 (1994); Keefer et al., Biol. Reprod., 50: 935-939 (1994); Sims et al., Proc. Natl. Acad. Sci., USA, 90: 6143-6147 (1993); WO97/07668; WO97/07669; WO94/26884; WO94/24274; as well as U.S. Pat. Nos. 4,944,384 and 5,057,420 (which describe bovine nuclear transplantation), all of which are incorporated in their entirety herein by reference.

Briefly, methods for nuclear transfer typically include the steps of: (1) enucleating an oocyte; (2) isolating a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop.

Oocytes are generally retrieved from deceased animals, although they may be isolated also from either oviducts and/or ovaries of live animals. Oocytes are typically matured in a variety of medium known to those of ordinary skill in the art prior to enucleation. Enucleation of the oocyte can be performed in a number of manners well known to those of ordinary skill in the art.

Insertion of the donor cell or nucleus into the enucleated oocyte to form a reconstituted cell is usually by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus.

A reconstituted cell is typically activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, are typically cultured in medium well known to those of ordinary skill in the art and then transferred to the womb of an animal.

Until recently, donor nuclei have been conventionally isolated almost entirely from primordial germ cells or embryonic cells. Indeed, until the late 1990s it was widely believed that only embryonic or undifferentiated cell types could direct any sort of fetal development following nuclear transfer. As a consequence most of today's techniques used in nuclear transfer procedures were developed utilising embryonic cells as donor cells and enucleated oocytes as recipient cells.

Notwithstanding, the isolation and use of embryonic donor cells requires specialised skills and is very labour intensive. More importantly, embryonic donor cells are a limited source of genetic material for nuclear transfer methods and their manipulation in vitro to produce cells, embryos, and animals whose genomes have been manipulated (e.g., transgenic) is not possible.

In 1997 this situation changed when it was reported that successful nuclear transfers had been done using cultured cell lines as donors (See, for example, Wilmut et al., Nature (London) 385, 810-183) (1997). Accordingly, with the advent of somatic cell nuclear transfer some of the problems with “traditional” embryonic cell nuclear transfer were solved. In particular, the limited source of genetic material was overcome. However, some problems still remain as not all techniques used in embryonic cell nuclear transfer can be readily utilised for somatic cell nuclear transfer. For example, due to the vastly different sizes of somatic cells compared to embryonic cells some of the techniques used traditionally are not readily adapted.

Indeed, the in vitro steps of the methods described above have low efficiency rates resulting in low pregnancy and calving rates, deaths after birth and developmental anomalies. The efficiency of live births from somatic cell nuclear transfer using the method described by Wilmut et al., (Wilmut et al., Nature 385: 810-183 (1997)) has been estimated to be approximately 1 out of 300, that is, the nuclear transfer efficiency is at best 0.4% (i.e. number of cloned lambs divided by the number of nuclear transfers used to produce that number of cloned lambs). More importantly, all of the methods described in the literature require highly skilled technicians and costly equipment. In order for the widespread practical application of nuclear transfer methods to become more commercially viable it is imperative that the cloning efficiency is increased, the costs associated with the methods decreased and the requirement for highly skilled technicians overcome.

Accordingly, despite the apparent establishment of many of the methods for somatic cell nuclear transfer there remain some major technical obstacles impeding the widespread practical application of these methods.

In an attempt to improve cloning efficiencies many research groups have modified the nuclear transfer methods; however, these modifications still require costly equipment and/or skilled labour. For example, a critical step in the nuclear transfer method outlined above is step 3; the step of inserting the donor cell or nucleus into the enucleated oocyte. As discussed above, this step typically requires two procedures, firstly, the microinjection of a donor cell under the zona pellucida of an enucleated oocyte and then secondly, fusion. However, the microinjection step impedes the commercialisation prospects of nuclear transfer as this requires specialised skills and equipment.

One technique that obviated the use of microinjection used previously in the more traditional approach with oocytes and embryonic donor cells involved the removal of the zona pellucida. See, for example, WO98/29532 and Peura et al. (1998), both of which are incorporated herein by reference. Unfortunately, an intact zona pellucida is generally considered important in somatic cell nuclear transfer for several reasons including (1) keeping the polar body close to the metaphase plate of the oocyte to indicate the appropriate site for enucleation, (2) keeping the donor cell close to the oocyte cytoplast before and during fusion, (3) providing protection for the pairs during fusion, and (4) supporting embryo development after reconstitution and activation. Accordingly, the technique of Peura et al. supra has not been successful used with somatic cells.

The presence of the zona pellucida during nuclear transfer means that sophisticated micromanipulation tools and high skill levels are required. In order to bypass the zona pellucida micromanipulators are used to transfer the donor cell into the perivitelline space of an enucleated oocyte to produce the reconstituted cell.

Micromanipulators are specialised devices that require tool making equipment including capillary pullers, grinders and microforges. More importantly, the use of the micromanipulators, and the equipment to make these, require skilled technicians. These requirements considerably limit the simplification needed for the large-scale application of nuclear transfer methods.

Based upon the foregoing, it can be seen that the benefits and prospects of somatic cell nuclear transfer procedures which provides for the use of donor cells which retain the ability to produce reconstituted cells capable of developing into viable animals and that provides for high cloning efficiency without the need for micromanipulators are considerable. Immediate consequences would include decreased costs of both equipment and labour, and would therefore lead to more cost effective cloned animal production.

To this end, the applicant has now developed s somatic cell nuclear transfer method which avoids the use of micromanipulators, thereby allowing for standard fusion techniques to be used, while maintaining or increasing cloning efficiency. In one embodiment, the method utilises zona pellucida-free, enucleated oocytes as recipients and somatic cells or nuclei as donors. To avoid unplanned embryo aggregation, the reconstituted zona pellucida-free embryos are cultured in specialised systems, either individually or as “aggregates” of two or three reconstituted nuclear transfer embryos, as conventional systems are inappropriate for the purpose.

### SUMMARY OF THE INVENTION

In the broadest aspect of the invention there is provided a novel and improved method for producing cloned mammalian cells.

Accordingly, in a first aspect the invention provides a method of nuclear transfer comprising the step of transferring a somatic cell or somatic cell nuclei into a zona pellucida-free, enucleated oocyte.

In a second aspect the invention provides a method for producing genetically engineered or transgenic mammals by which a desired gene is inserted, removed or modified in a somatic cell or cell nucleus prior to transferring the somatic cell or cell nucleus into a zona pellucida-free, enucleated oocyte.

The invention further provides a method for producing a genetically engineered or transgenic mammal comprising:

(i) inserting, removing or modifying a desired gene or genes in a somatic cell or cell nucleus;

(ii) inserting the somatic cell or cell nucleus into a zona pellucida-free, enucleated oocyte under conditions suitable for the formation of a reconstituted cell;

(iii) activating the reconstituted cell to form an embryo;

(iv) culturing said embryo until greater than the 2-cell developmental stage; and

(v) transferring said cultured embryo to a host mammal such that the embryo develops into a transgenic fetus.

In a third aspect, the present invention provides a method for cloning a mammal comprising:

(i) inserting a desired somatic cell or cell nucleus into a zona pellucida-free, enucleated mammalian oocyte, under conditions suitable for the formation of a reconstituted cell;

(ii) activating the reconstituted cell to form an embryo;

(iii) culturing said embryo until greater than the 2-cell developmental stage; and

(iv) transferring said cultured embryo to a host mammal such that the embryo develops into a fetus.

Also provided by the present invention are mammals obtained according to the above method, and offspring of those mammals.

Oocytes may be isolated from any mammal by known procedures. For example, oocytes can be isolated from either oviducts and/or ovaries of live animals by oviductal recovery procedures or transvaginal oocyte recovery procedures well known in the art and described herein. Furthermore, oocytes can be isolated from deceased animals. For example, ovaries can be obtained from abattoirs and the oocytes aspirated from these ovaries. The oocytes can also be isolated from the ovaries of a recently sacrificed animal or when the ovary has been frozen and/or thawed. Preferably, the oocytes are freshly isolated from the oviducts.

Oocytes or cytoplasts may also be cryopreserved before use.

While the methods described herein are useful for nuclear transfer in any mammal, it is particularly useful for ungulates. Preferably, the ungulate is selected from the group consisting of domestic or wild representatives of bovids, ovids, cervids, suids, equids and camelids. Examples of such representatives are cows or bulls, bison, buffalo, sheep, big-horn sheep, horses, ponies, donkeys, mule, deer, elk, caribou, goat, water buffalo, camels, llama, alpaca, and pigs. Especially preferred in the bovine species are Bos taurus, Bos indicus, and Bos buffaloes cows or bulls.

Removal of the zona pellucida can be accomplished by any known procedure. Preferably, the step of removing the zona pellucida is selected from the group consisting of physical manipulation, chemical treatment and enzymatic digestion. More preferably, the zona pellucida is removed by enzymatic digestion. Preferably, the enzyme used to digest the zona pellucida is a protease, a pronase or a combination thereof. More preferably, the enzyme is a pronase.

Preferably, the pronase is used at a concentration between 0.1 to 5%. More preferably, the concentration is between 0.25% to 2%. Most preferably, the pronase is at a concentration of about 0.5%.

It will be appreciated by those skilled in the art that any procedure of enucleation of the oocyte can be performed, including, aspiration, physical removal, use of DNA-specific fluorochromes, and irradiation with ultraviolet light. Preferably, the enucleation is by physical means. Most preferable, the physical means is bisection.

Somatic cells are selected from the group consisting of epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells.

These may be obtained from different organs, e.g., skin, lung, pancreas, liver, stomach, intestine, heart, reproductive organs, bladder, kidney, urethra and other urinary organs, etc.

Preferably, the somatic cells are fibroblast cells or granulosa cells. Most preferably, the somatic cells are in vitro cultured fibroblasts or granulosa cells.

Preferably, the step of transferring the somatic cell or nucleus is by fusion. More preferably, the method of fusion is selected from the group consisting of chemical fusion, electrofusion and biofusion. Preferably, the chemical fusion or biofusion is accomplished by exposing the zona pellucida-free, enucleated oocyte and somatic cell combination to a fusion agent. Preferably, the fusion agent is any compound or biological organism that can increase the probability that portions of plasma membranes from different cells will fuse when somatic cell donor is placed adjacent to the zona pellucida-free, enucleated oocyte recipient. Most preferably, the fusion agents are selected from the group consisting of polyethylene glycol (PEG), trypsin, dimethylsulfoxide (DMSO), lectins, agglutinin, viruses, and Sendai virus.

The electrofusion is preferably induced by application of an electrical pulse across the contact/fusion plane. More preferably, the electrofusion comprises the step of delivering one or more electrical pulses to the zona pellucida-free, enucleated oocyte and somatic cell combination.

In a preferred embodiment, the method of the invention comprises a further step of increasing the cytoplasmic volume of a reconstituted cell by fusing the reconstituted cell with one or more oocyte(s).

Accordingly, in a fourth aspect the present invention provides a method for cloning a mammal comprising:

(i) inserting a desired somatic cell or cell nucleus into a first zona pellucida-free, enucleated mammalian oocyte, under conditions suitable for the formation of a reconstituted cell;

(ii) fusing a second oocyte to said reconstituted cell thereby increasing the cytoplasmic volume;

(iii) activating the reconstituted cell to form an embryo;

(iv) culturing said embryo until greater than the 2-cell developmental stage; and

(v) transferring said cultured embryo to a host mammal such that the embryo develops into a fetus.

Steps (i) and (ii) may be undertaken separately or simultaneously.

Alternatively, in one preferred embodiment, there is provided a method of culturing a reconstituted cell (embryo) comprising

(i) inserting a desired somatic cell or cell nucleus into a zona pellucida-free, enucleated mammalian oocyte, under conditions suitable for the formation of a reconstituted cell;

(ii) activating the reconstituted cell

(iii) incubating and culturing one or more of said cells until embryos of greater than 2 cells develop.

Preferably, the embryos are cultured until greater than 60 cells. More preferably, between 60 to 200 cells.

Preferably, two or more cells are cultured together, more preferably, two or three cells are cultured together.

In an alternative embodiment, single reconstituted cells are cultured until embryos of between 8 to 128 cells are produced and then 2 or more embryos are combined and cultured together as aggregates.

Culturing zona pellucida-free nuclear transfer embryos as aggregates, increases the cell numbers in the final embryos for transfer and increases pregnancy rates.

Also provided by the present invention are mammals obtained according to the above methods, and offspring of those mammals.

### BRIEF DESCRIPTION OF THE FIGURES

FIG. 1 shows the results of a preferred method according to the present invention. The procedure and result of zona pellucida-free, oocyte-somatic cell transfer is provided. Panel A shows oocytes aligned in a petri dish prior to bisection. Panel B shows manual bisection of oocytes to produce enucleated oocytes (cytoplasts) and karyoplasts. Panel C shows enucleated oocyte and somatic cell prior to attachment. Panel D shows enucleated oocyte-somatic cell after attachment, prior to fusion. Panel E shows enucleated oocyte-somatic cell pair aligned on the electrofusion wire for the first fusion. Panel F shows unfused enucleated oocytes and fused enucleated oocyte-somatic cell pairs aligned to the electrofusion wire for the second fusion. Panel G shows blastocyst developed in the GO system 7 days after fusion. Panel H shows the same blastocyst after removal from the GO system.

### DETAILED DESCRIPTION OF THE INVENTION

The practice of the present invention employs, unless otherwise indicated, conventional molecular biology, cellular biology, and cloning techniques within the skill of the art. Such techniques are well known to the skilled worker, and are explained fully in the literature. See, for example, Sambrook and Russell “Molecular Cloning: A Laboratory Manual” (2001); Cloning: A Practical Approach,” Volumes I and II (D. N. Glover, ed., 1985); “Antibodies: A Laboratory Manual” (Harlow & Lane, eds., 1988); “Animal Cell Culture” (R. I. Freshney, ed., 1986); “Immobilised Cells and Enzymes” (IRL Press, 1986).

Before the present methods are described, it is understood that this invention is not limited to the particular materials and methods described, as these may vary. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “an,” and “the” include plural reference unless the context clearly dictates otherwise. Thus, for example, a reference to “a somatic cell” includes a plurality of such cells, and a reference to “an oocyte” is a reference to one or more oocytes, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

All publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

The present invention provides improved procedures for cloning mammals by nuclear transfer or nuclear transplantation. In the subject application, the terms “nuclear transfer” or “nuclear transplantation” are used interchangeably; however, these terms as used herein refers to introducing a full complement of nuclear DNA from one cell to an enucleated cell.

The first step in the preferred methods involves the isolation of a recipient oocyte from a suitable animal. In this regard, the oocyte may be obtained from any animal source and at any stage of maturation. Suitable mammalian sources include members of the Orders Primates, Rodentia, Lagomorpha, Cetacea, Carnivora, Perissodactyla and Artiodactyla. Members of the Orders Perissodactyla and Artiodactyla are particularly preferred because of their similar biology and economic importance.

For example, Artiodactyla comprises approximately 150 living species distributed through nine families: pigs (Suidae), peccaries (Tayassuidae), hippopotamuses (Hippopotamidae), camels (Camelidae), chevrotains (Tragulidae), giraffes and okapi (Giraffidae), deer (Cervidae), pronghom (Antilocapridae), and cattle, sheep, goats and antelope (Bovidae). Many of these animals are used as feed animals in various countries. More importantly, with respect to the present invention, many of the economically important animals such as goats, sheep, cattle and pigs have very similar biology and share high degrees of genomic homology.

The Order Perissodactyla comprises horses and donkeys, which are both economically important and closely related. Indeed, it is well known that horses and donkeys interbreed.

In one embodiment, the oocytes will be obtained from ungulates, and in particular, bovids, ovids, cervids, suids, equids and camelids. Examples of such representatives are cows or bulls, bison, buffalo, sheep, big-horn sheep, horses, ponies, donkeys, mule, deer, elk, caribou, goat, water buffalo, camels, llama, alpaca, and pigs. Especially preferred in the bovine species are Bos taurus, Bos indicus, and Bos buffaloes cows or bulls.

Methods for isolation of oocytes are well known in the art. For example, oocytes can be isolated from either oviducts and/or ovaries of live animals by oviductal recovery procedures or transvaginal oocyte recovery procedures well known in the art. See, for example, Pieterse et al., 1988, “Aspiration of bovine oocytes during transvaginal ultrasound scanning of the ovaries,” Theriogenology 30: 751-762. Furthermore, oocytes can be isolated from ovaries or oviducts of deceased animals. For example, ovaries can be obtained from abattoirs and the oocytes aspirated from these ovaries. The oocytes can also be isolated from the ovaries of a recently sacrificed animal or when the ovary has been frozen and/or thawed.

Briefly, in one preferred embodiment, immature (prophase I) oocytes from mammalian ovaries are harvested by aspiration. For the successful use of techniques such as genetic engineering, nuclear transfer and cloning, once these oocytes have been harvested they must generally be matured in vitro before these cells may be used as recipient cells for nuclear transfer.

The stage of maturation of the oocyte at enucleation and nuclear transfer has been reported to be significant to the success of nuclear transfer methods. (See, for example, Prather et al., Differentiation, 48, 1-8, 1991). In general, successful mammalian embryo cloning practices use the metaphase II stage oocyte as the recipient oocyte because at this stage it is believed that the oocyte can be or is sufficiently activated to treat the introduced nucleus as it does a fertilising sperm.

The in vitro maturation of oocytes usually takes place in a maturation medium until the oocyte have extruded the first polar body, or until the oocyte has attained the metaphase II stage. In domestic animals, and especially cattle, the oocyte maturation period generally ranges from about 16-52 hours, preferably about 28-42 hours and more preferably about 18-24 hours post-aspiration. For purposes of the present invention, this period of time is known as the “maturation period.”

Oocytes can be matured in a variety ways and using a variety of media well known to a person of ordinary skill in the art. See, for example, U.S. Pat. No. 5,057,420; Saito et al., 1992, Roux's Arch. Dev. Biol. 201: 134-141 for bovine organisms and Wells et al., 1997, Biol. Repr. 57: 385-393 for ovine organisms and WO97/07668, entitled “Unactivated Oocytes as Cytoplast Recipients for Nuclear Transfer,” all hereby incorporated herein by reference in the entirety, including all figures, tables, and drawings.

One of the most common media used for the collection and maturation of oocytes is Tissue Culture Medium-199 (TCM-199), and 1 to 20% serum supplement including fetal calf serum (FCS), newborn serum, estrual or non-estrual cow serum, lamb serum or steer serum. Example 1 of the present application shows one example of a preferred maintenance medium TCM-199 with Earl salts supplemented with 15% cow serum and including 10 IU/ml pregnant mare serum gonadotropin and 5 IU/ml human chorionic gonadotropin (Suigonan® Vet, Intervet, Australia). Oocytes can be successfully matured in this type of medium within an environment comprising 5% CO₂ at 39° C.

While it will be appreciated by those skilled in the art that freshly isolated and matured oocytes are preferred, it will also be appreciated that it is possible to cryopreserve the oocytes after harvesting or after maturation. Accordingly, the term “cryopreserving” as used herein can refer to freezing an oocyte, cytoplast, a cell, embryo, or animal of the invention. The oocytes, cytoplast, cells, embryos, or portions of animals of the invention are frozen at temperatures preferably lower than 0° C., more preferably lower than −80° C., and most preferably at temperatures lower than −196° C. Oocytes, cells and embryos of the invention can be cryopreserved for an indefinite amount of time. It is known that biological materials can be cryopreserved for more than fifty years. For example, semen that is cryopreserved for more than fifty years can be utilised to artificially inseminate a female bovine animal. Methods and tools for cryopreservation are well known to those skilled in the art. See, for example, U.S. Pat. No. 5,160,312, entitled “Cryopreservation Process for Direct Transfer of Embryos.”

If cryopreserved oocytes are utilised then these must be initially thawed before placing the oocytes in maturation medium. Methods of thawing cryopreserved materials such that they are active after the thawing process are well-known to those of ordinary skill in the art.

In a further preferred embodiment, mature (metaphase II) oocytes, which have been matured in vivo, are harvested and used in the nuclear transfer methods disclosed herein. Essentially, mature metaphase II oocytes are collected surgically from either non-superovulated or superovulated mammals 35 to 48 hours past the onset of estrus or past the injection of human chorionic gonadotropin (hCG) or similar hormone.

Where oocytes have been cultured in vitro cumulus cells that may have accumulated may be removed to provide oocytes that are at a more suitable stage of maturation for enucleation. Cumulus cells may be removed by pipetting or vortexing, for example, in the presence of 0.5% hyaluronidase.

After the maturation period as described above the zona pellucida can then removed from the oocytes; however, in one particularly preferred embodiment, prior to the removal of the zona pellucida, the oocytes are placed in phosphate-buffered saline (PBS) containing 200 μg/ml phytohemagglutanin (PHA) so that the polar body (PB) attaches to the oocyte. It has been shown that the chromosome containing metaphase plate is adjacent to the PB in over 90% of cases (Peura et al, 1998). After zona removal and oocyte bisection, the karyplast (nucleus containing “half” of the bisected oocyte) can be easily identified and discarded.

The advantages of zona pellucida removal include providing a simpler, quicker and cheaper nuclear transfer method. In addition, the removal of the zona pellucida allows for large-scale production of nuclear transfer embryos. The removal of the zona pellucida from the oocyte may be carried out by any method known in the art including physical manipulation (mechanical opening), chemical treatment or enzymatic digestion (Wells and Powell, 2000). Physical manipulation may involve the use of a micropipette or a microsurgical blade. Preferably, enzymatic digestion is used.

In one particularly preferred embodiment, the zona pellucida is removed by enzymatic digestion in the presence of a protease or pronase. Briefly, mature oocytes are placed into a solution comprising a protease, pronase or combination of each at a total concentration in the range of 0.1%-5%, more preferably 0.25%-2% and most preferably about 0.5%. The mature oocyte is then allowed to incubate at between 30° C. to about 45° C., preferably about 39° C. for a period of 1 to 30 minutes. Preferably the oocytes are exposed to the enzyme for about 5 minutes. Although pronase may be harmful to the membranes of oocytes, this effect may be minimised by addition of serum such as FCS or cow serum. The unique advantage of zona pellucida removal with pronase is that no individual treatment is required, and the procedure can be performed in quantities of 100's of oocytes. Once the zona pellucida has been removed the zona pellucida-free mature oocyte may be rinsed in 4 ml Hepes buffered TCM-199 medium supplemented with 20% FCS and 10 μg/ml cytochalasin B and then enucleated.

The terms “enucleation”, “enucleated” and “enucleated oocyte” are used interchangeably herein and refers to an oocyte which has had part of its contents removed.

Enucleation of the oocyte may be achieved physically, by actual removal of the nucleus, pronuclei or metaphase plate (depending on the oocyte), or functionally, such as by the application of ultraviolet radiation or another enucleating influence. All of these methods are well known to those of ordinary skill in the art. For example, physical means includes aspiration (Smith & Wilmut, Biol. Reprod., 40: 1027-1035 (1989)); functional means include use of DNA-specific fluorochromes (See, for example, Tusnoda et al., J. Reprod. Fertil. 82: 173 (1988)), and irradiation with ultraviolet light (See, for example, Gurdon, Q. J. Microsc. Soc., 101: 299-311 (1960)). Enucleation may also be effected by other methods known in the art. See, for example, U.S. Pat. No. 4,994,384; U.S. Pat. No. 5,057,420; and Willadsen, 1986, Nature 320:63-65, all of which are incorporated herein by reference.

Preferably, the oocyte is enucleated by means of manual bisection. Oocyte bisection may be carried out by any method known to those skilled in the art. In one preferred embodiment, the bisection is carried out using a microsurgical blade as described in WO98/29532 which is incorporated by reference herein. Briefly, oocytes are split asymmetrically into fragments representing approximately 30% and 70% of the total oocyte volume using an ultra sharp splitting blade (AB Technology, Pullman, Wash., USA). The oocytes may then be screened to identify those of which have been successfully enucleated. This screening may be effected by selecting that bisected “half” with the polar body attached or by staining the oocytes with 1 microgram per millilitre of the Hoechst fluorochrome 33342 dissolved in TCM-199 media supplemented with 20% FCS, and then viewing the oocytes under ultraviolet irradiation with an inverted microscope for less than 10 seconds. The oocytes that have been successfully enucleated (demi-oocytes) can then be placed in a suitable culture medium, e.g., TCM-199 media supplemented with 20% FCS.

In the present invention, the recipient oocytes will preferably be enucleated at a time ranging from about 10 hours to about 40 hours after the initiation of in vitro maturation, more preferably from about 16 hours to about 24 hours after initiation of in vitro maturation, and most preferably about 16-20 hours after initiation of in vitro maturation.

The bisection technique described herein requires much less time and skill than other methods of enucleation and the subsequent selection by staining results in high accuracy. Consequently, for large-scale application of cloning technology the present bisection technique can be more efficient than other techniques.

A single mammalian somatic cell of the same species as the enucleated oocyte can then be transferred by fusion into the enucleated oocyte thereby producing a reconstituted cell.

The term “somatic cell” as used herein is taken to mean any cell from an animal at any stage of development, other than an embryonic cell or germ cell.

According to the invention, cell nuclei derived from differentiated fetal or adult somatic cells are transferred into zona pellucida-free, enucleated oocytes of the same species as the donor nuclei. Differentiated somatic cells are those cells that are past the early embryonic stage. More particularly, the differentiated cells are those from at least past the embryonic disc stage (day 10 of bovine embryogenesis). The differentiated cells may be derived from ectoderm, mesoderm or endoderm.

Mammalian somatic cells may be obtained by well-known methods. See, for example, U.S. Pat. No. 5,945,577, which teaches nuclear transfers from differentiated donor somatic cells to enucleated oocytes and U.S. Pat. No. 6,022,197, which teaches that fibroblasts from a fibroblast cell culture derived from an adult ear punch may be used as nuclear donors in a nuclear transfer process, both of these references are incorporated herein by reference.

It is preferred that the donor somatic cells of the present invention be induced to quiescence prior to fusion into the recipient zona pellucida-free, enucleated oocyte. In accordance with the teachings of PCT/GB96/02099 and WO97/07668, both assigned to the Roslin Institute (Edinburgh), it is preferred that the donor nucleus be in either the G0 or G1 phase of the cell cycle at the time of transfer. Donors must be diploid at the time of transfer in order to maintain correct ploidy of the reconstituted cell.

Mammalian somatic cells useful in the present invention include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells, i.e., cells useful in the subject invention, may be obtained from any cell or organ of the body.

A particularly preferred donor cell is the fibroblast or fibroblast-like cell. Fibroblast cells are an ideal cell type because they can be obtained from developing fetuses and adult animals in large quantities. Importantly, these cells can be easily propagated in vitro with a rapid doubling time and can be clonally propagated for use in gene targeting procedures.

Fibroblast cells may be collected from an ear skin biopsy and cut into small pieces (3 mm²) and cultured. A variety of methods for culturing cells exist in the art. See, for example, Culture of Animal Cells; A manual of Basic Technique (2^{nd}. edition), Freshney, copyright 1987, Alan R. Liss, Inc., New York. In one particularly preferred embodiment, explant cells from a skin biopsy are cultured in TCM-199 medium plus 20% FCS and antibiotics at 37° C., in a humidified atmosphere of 5% CO₂ and 95% air. After a week in culture, fibroblast cell monolayers form around the tissue explants. The explants are then removed to start new culture and the fibroblast cells are harvested by incubation with 0.05% trypsin for 5 min. The trypsin is then inactivated by the addition of 800 μl TCM-199 medium and 20% FCS. For long term storage, the cultured cells may be collected following trypsin treatment, frozen in 10% dimethyl sulfoxide and stored in liquid nitrogen. Upon use for nuclear transfer, cells are thawed and cultured to confluency for passage. For each passage (estimated 2 cell doublings per passage), cells are cultured until confluent, disaggregated by incubation in a 0.1% (w/v) trypsin and EDTA solution for 1 min at 37° C. and allocated to three new flasks for further passaging. Normally, each passage lasts about 6 days.

Confirmation of fibroblast phenotype of donor cells may be conducted by immunocytochemical staining with monoclonal antibodies directed against the cytoskeletal filaments vimentin (for fibroblasts) or cytokeratin (for epithelial cells). In a preferred confirmation protocol, cells are grown to confluency. Cells are then washed with phosphate buffered saline (PBS) and fixed in methanol at 4° C. for 20 minutes. After fixation the cells are washed in PBS and blocked with 3% bovine serum albumin (BSA) in PBS for 15 min at 37° C. The block is removed and 100 μl of either a 1:40 dilution anti-vimentin clone V9 (Sigma, cat#6630) or a 1:400 dilution of anti-pan cytokeratin clone-11 (Sigma, cat#2931) is added. Cells are incubated for 1 h at 37° C., washed with PBS and incubated for 1 h with 100 μl of a 1:300 dilution of FITC-labelled anti-mouse IgG. Cells are washed in PBS, covered with 50% glycerol in PBS under a coverslip and observed by fluorescence microscopy. Appropriate controls for auto-fluorescence and secondary antibodies should be included.

Analysis of cell cycle stage may be performed as described in Kubota et al., PNAS 97: 990-995 (2000). Briefly, cell cultures at different passages are grown to confluency. After trypsinisation, cells are washed with TCM-199 medium plus 10% FCS and re-suspended to a concentration of 5×10⁵ cells/ml in 1 ml PBS with glucose (6.1 mM) at 4° C. Cells are fixed overnight by adding 3 ml of ice-cold ethanol. For nuclear staining, cells are then pelleted, washed with PBS and re-suspended in PBS containing 30 μg/ml propidium iodide and 0.3 mg/ml RNase A. Cells are allowed to incubate for 1 h at room temperature in the dark before filtered through a 30 μm mesh. Cells are then analyzed.

To examine the ploidy of the cultured somatic donor cells at various passages, chromosome counts may be determined at different passages of culture using standard preparation of metaphase spreads (See, for example, Kubota et al., PNAS 97: 990-995 (2000)).

Cultured donor cells may also be genetically altered by transgenic methods well-known to those of ordinary skill in the art. See, for example, Molecular Cloning a Laboratory Manual, 2nd Ed., 1989, Sambrook, Fritsch and Maniatis, Cold Spring Harbor Laboratory Press; U.S. Pat. No. 5,612,205; U.S. Pat. No. 5,633,067; EPO 264 166, entitled “Transgenic Animals Secreting Desired Proteins Into Milk”; WO94/19935, entitled “Isolation of Components of Interest From Milk”; WO93/22432, entitled “Method for Identifying Transgenic Pre-implantation Embryos”; and WO95/175085, entitled “Transgenic Production of Antibodies in Milk,” all of which are incorporated by reference herein in their entirety including all figures, drawings and tables. Any known method for inserting, deleting or modifying a desired gene from a mammalian cell may be used for altering the differentiated cell to be used as the nuclear donor. These procedures may remove all or part of a gene, and the gene may be heterologous. Included is the technique of homologous recombination, which allows the insertion, deletion or modification of a gene or genes at a specific site or sites in the cell genome.

Examples for modifying a target DNA genome by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

The present invention can thus be used to provide adult mammals with desired genotypes. Multiplication of adult ungulates with proven genetic superiority or other desirable traits is particularly useful, including transgenic or genetically engineered animals, and chimeric animals. Furthermore, cell and tissues from the nuclear transfer fetus, including transgenic and/or chimeric fetuses, can be used in cell, tissue and organ transplantation.

Methods for generating transgenic cells typically include the steps of (1) assembling a suitable DNA construct useful for inserting a specific DNA sequence into the nuclear genome of a cell; (2) transfecting the DNA construct into the cells; (3) allowing random insertion and/or homologous recombination to occur. The modification resulting from this process may be the insertion of a suitable DNA construct(s) into the target genome; deletion of DNA from the target genome; and/or mutation of the target genome.

DNA constructs can comprise a gene of interest as well as a variety of elements including regulatory promoters, insulators, enhancers, and repressors as well as elements for ribosomal binding to the RNA transcribed from the DNA construct.

DNA constructs can also encode ribozymes and anti-sense DNA and/or PNA. These examples are well known to a person of ordinary skill in the art and are not meant to be limiting.

Due to the effective recombinant DNA techniques available in conjunction with DNA sequences for regulatory elements and genes readily available in data bases and the commercial sector, a person of ordinary skill in the art can readily generate a DNA construct appropriate for establishing transgenic cells using the materials and methods described herein.

Transfection techniques are well known to a person of ordinary skill in the art and materials and methods for carrying out transfection of DNA constructs into cells are commercially available. Materials typically used to transfect cells with DNA constructs are lipophilic compounds, such as Lipofectin™ for example. Particular lipophilic compounds can be induced to form liposomes for mediating transfection of the DNA construct into the cells.

Target sequences from the DNA construct can be inserted into specific regions of the nuclear genome by rational design of the DNA construct. These design techniques and methods are well known to a person of ordinary skill in the art. See, for example, U.S. Pat. No. 5,633,067; U.S. Pat. No. 5,612,205 and WO93/22432, all of which are incorporated by reference herein in their entirety. Once the desired DNA sequence is inserted into the nuclear genome, the location of the insertion region as well as the frequency with which the desired DNA sequence has inserted into the nuclear genome can be identified by methods well known to those skilled in the art.

Once the transgene is inserted into the nuclear genome of the donor somatic cell, that cell, like other donor somatic cells of the invention, can be used as a nuclear donor in the nuclear transfer methods disclosed herein. The means of transferring the nucleus of a somatic cell into the zona pellucida-free, enucleated oocyte preferably involves cell fusion to form a reconstituted cell.

Fusion is typically induced by application of a direct current (DC) electrical pulse across the contact/fusion plane, but additional alternating current (AC) may be used to assist alignment of donor and recipient cells. Electrofusion produces a pulse of electricity that is sufficient to cause a transient breakdown of the plasma membrane and which is short enough that the membrane reforms rapidly. Thus, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384 by Prather et al., (incorporated by reference in its entirety herein) for a further discussion of this process. A variety of electrofusion media can be used including e.g., sucrose, mannitol, sorbitol and phosphate buffered solution.

Fusion can also be accomplished using Sendai virus as a fusogenic agent (Graham, Wister Inot. Symp. Monogr., 9, 19, 1969). Fusion may also be induced by exposure of the cells to fusion-promoting chemicals, such as polyethylene glycol.

Preferably, the donor somatic cell and zona pellucida-free, enucleated oocyte are placed in a 500 μm fusion chamber and covered with 4 ml of 26° C.-27° C. fusion medium (0.3M mannitol, 0.1 mM MgSO₄, 0.05 mM CaCl₂). The cells are then electrofused by application of a double DC electrical pulse of 70-100V for about 15 μs, approximately 1 s apart. After fusion, the resultant fused reconstituted cells are then placed in a suitable medium until activation, e.g., TCM-199 medium.

In a preferred method of cell fusion the donor somatic cell is firstly attached to the zona pellucida-free, enucleated oocyte. For example, a compound is selected to attach the somatic cell to the zona pellucida-free, enucleated oocyte to enable fusing of the somatic cell and zona pellucida-free, enucleated oocyte membranes. The compound may be any compound capable of agglutinating cells. The compound may be a protein or glycoprotein capable of binding or agglutinating carbohydrate. More preferably the compound is a lectin. The lectin may be selected from the group consisting of Concanavalin A, Canavalin A, Ricin, soybean lectin, lotus seed lectin and phytohemaglutinin (PHA). Preferably the compound is PHA.

Preferably the zona pellucida-free, enucleated oocytes are exposed to PHA before being contacted with a somatic cell. Preferably the zona pellucida-free, enucleated oocytes are exposed to a concentration of PHA in the range of 50-400 μg/ml. Most preferably the concentration is about 200 μg/ml. The zona pellucida-free, enucleated oocytes may be exposed to PHA from 1-60 s. Most preferably the enucleated oocytes are exposed to PHA for 3 s.

Following treatment with PHA, the zona pellucida-free, enucleated oocyte may be contacted with a somatic cell to attach said somatic cell to the zona pellucida-free, enucleated oocyte. The zona pellucida-free, enucleated oocyte may be contacted with a somatic cell by conventional methods known to those skilled in the field. Preferably the zona pellucida-free, enucleated oocyte is contacted with a somatic cell by manipulation using a micropipette. The zona pellucida-free, enucleated oocyte and attached somatic cell then may be fused as described above.

In one preferred embodiment, the method of electrofusion described above also comprises a further fusion step, or the fusion step described above comprises one donor somatic cell and two or more zona pellucida-free, enucleated oocytes. The double fusion method has the advantageous effect of increasing the cytoplasmic volume of the reconstituted cell.

A reconstituted cell is typically activated by electrical and/or non-electrical means before, during, and/or after fusion of the nuclear donor and recipient oocyte (See, for example, Susko-Parrish et al., U.S. Pat. No. 5,496,720). Activation methods include:
1). Electric pulses;
2). Chemically induced shock;
3). Penetration by sperm;
4). Increasing levels of divalent cations in the oocyte by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators; and
5). Reducing phosphorylation of cellular proteins in the oocyte by known methods, e.g., by the addition of kinase inhibitors, e.g., serine-threonine kinase inhibitors, such as 6-dimethyl-aminopurine, staurosporine, 2-aminopurine, and sphingosine. Alternatively, phosphorylation of cellular proteins may be inhibited by introduction of a phosphatase into the oocyte, e.g., phosphatase 2A and phosphatase 2B.

The reconstituted cell may also be activated by known methods. Such methods include, e.g., culturing the reconstituted cell at sub-physiological temperature, in essence by applying a cold, or actually cool temperature shock to the reconstituted cell. This may be most conveniently done by culturing the reconstituted cell at room temperature, which is cold relative to the physiological temperature conditions to which embryos are normally exposed. Suitable oocyte activation methods are the subject of U.S. Pat. No. 5,496,720, to Susko-Parrish et al., herein incorporated by reference in its entirety.

The activated reconstituted cells may then be cultured in a suitable in vitro culture medium until the generation of cells and cell colonies. Culture media suitable for culturing and maturation of embryos are well known in the art. Examples of known media, which may be used for bovine embryo culture and maintenance, include Ham's F-10 plus 10% FCS, TCM-199 plus 10% FCS, Tyrodes-Albumin-Lactate-Pyruvate (TALP), Dulbecco's Phosphate Buffered Saline (PBS), synthetic oviductal fluid (“SOF”), B2, CR1aa medium and high potassium simplex medium (“KSOM”), Eagle's and Whitten's media. One of the most common media used for the collection and maturation of oocytes is TCM-199, and 1 to 20% serum supplement including FCS, newborn serum, estrual cow serum, lamb serum or steer serum. A preferred maintenance medium includes TCM-199 with Earl salts, 10% FSC, 0.2 mM Na pyruvate and 50 μg/ml gentamicin sulphate. Any of the above may also involve co-culture with a variety of cell types such as granulosa cells, oviduct cells, BRL cells and uterine cells and STO cells. Alternatively, in one preferred embodiment, there is provided a method of culturing a reconstituted cell (embryo) comprising

(i) inserting a desired somatic cell or cell nucleus into a zona pellucida-free, enucleated mammalian oocyte, under conditions suitable for the formation of a reconstituted cell;

(ii) activating the reconstituted cell

(iii) incubating and culturing one or more of said cells until embryos of greater than 2 cells develop.

Preferably, the embryos are cultured until greater than 60 cells. More preferably, between 60 to 200 cells.

Briefly, the above method is described as the Well of Well (WOW) system. This method involves culturing reconstituted cells either individually or groups in small depressions (“V” or “U” shaped) made on the bottom of the dish by pressing the ground tip of “darning” type needles (such as the “Aggregation needles” manufactured and supplied by BLS Ltd., Budapest, Hungary, Catalogue no. DN-09) into the bottom of the culture dish, which is typically a 4 well “Nunclone” dish (Vajta et al, 2000.). These depressions (WOWs) are typically 0.5 to 2 mm deep and 0.5 to 2 mm in diameter at the top. After activation the embryos are placed into these WOW depressions, either individually, or 2 or 3 “reconstituted” nuclear transfer embryos can be placed together in each WOW and cultured as aggregates after activation (when they are still single cells, prior to cell division). Alternatively the reconstituted single cell nuclear transfer embryos can be cultured individually in the WOWs for 3 to 4 days (when they are typically between 8 and 128 cells) and then 2 or 3 such embryos can be combined in single wells for further culture as “aggregates”. Culturing nuclear transfer embryos as such aggregates, increases the cell numbers in the final embryos for transfer and increases pregnancy rates (Peura et al, 1998).

In one further embodiment, there is provided a method of culturing a reconstituted cell (embryo) comprising providing a reconstituted cell (embryo) according to the methods as hereinbefore described in medium;

obtaining a tube having at least two open ends and wherein one end is capable of receiving the reconstituted cell (embryo), the tube having a diameter capable of drawing and maintaining the reconstituted cell (embryo) in the medium in the tube;

drawing the reconstituted cell (embryo) into the tube; and

incubating and culturing the reconstituted cell (embryo) in the tube.

In the method of culturing in the tube, the tube is preferably of a grade which poses minimal toxicity to the embryo. Most preferably, it is uncoated or treated, acid washed borosilicate laboratory grade glass. Most importantly, the tube must have holding capacity such that the medium surrounding the embryo is held in the tube generally by capillary action and surface tension so as to maintain the embryo within the tube. The media is cushioned in the tube by an air/media interface from either end of the tube, and occasionally by a small plug of oil.

The tube of the culture system may be of any diameter providing that it can hold and maintain an embryo in the medium within the tube so that the embryo may develop within the drawn medium. Therefore, the tube must be capable of providing sufficient capillary action and surface tension to the medium to maintain the medium vertically within the tube and also to draw the embryo up the tube. Preferably, the tube has an internal diameter of 200-250 mm. The tube may be a capillary tube. Narrower ranges of internal diameter may be in the order of 200 mm or less with the size of an embryo being the limiting factor. Narrower ranges such as 200 mm or less may be of benefit for full promotion of development.

The embryo may be drawn or taken up into the tube under passive capillary action or by an active pressure drawing the fluid up the tube. The second method may be employed providing the tube has sufficient capability to maintain and hold the medium and embryo in the tube. Once the embryo is drawn into the tube, it is preferred that the tube is held vertically rather than horizontally so as to create a cushion on the air/medium interface. Horizontal incubation may also be employed although the vertical orientation is most preferred.

The culture tube system is designed so that the embryo contained therein can develop to a further advanced stage of development, preferably to the mature blastocyst stage. However, any stages such as early cleavage or morula may be selected after observing the development of the embryo directly in the tube. The embryo can be removed at any time depending on the desired development stage. This has many advantages since the embryo, once contained and matured in the culture system, is immediately available with minimal manipulation for implantation into an animal at a suitable stage of development.

Afterward, the cultured reconstituted cell or embryos are preferably washed and then placed in a suitable media, e.g., TCM-199 medium containing 10% FCS contained in well plates which preferably contain a suitable confluent feeder layer. Suitable feeder layers include, by way of example, fibroblasts and epithelial cells, e.g., fibroblasts and uterine epithelial cells derived from ungulates, chicken fibroblasts, murine (e.g., mouse or rat) fibroblasts, STO and SI-m220 feeder cell lines, and BRL cells.

In one embodiment, the feeder cells comprise mouse embryonic fibroblasts. Preparation of a suitable fibroblast feeder layers are well known in the art.

The reconstituted cells are cultured on the feeder layer until the reconstituted cells reach a size suitable for transferring to a recipient female, or for obtaining cells which may be used to produce cells or cell colonies. Preferably, these reconstituted cells will be cultured until at least about 2 to 400 cells, more preferably about 4 to 128 cells, and most preferably at least about 50 cells. The culturing will be effected under suitable conditions, i.e., about 39° C. and 5% CO₂, with the culture medium changed in order to optimise growth typically about every 2-5 days, preferably about every 3 days.

The methods for embryo transfer and recipient animal management in the present invention are standard procedures used in the embryo transfer industry. Synchronous transfers are important for success of the present invention, i.e., the stage of the nuclear transfer embryo is in synchrony with the estrus cycle of the recipient female. This advantage and how to maintain recipients are reviewed in Siedel, G. E., Jr. (“Critical review of embryo transfer procedures with cattle” in Fertilization and Embryonic Development in Vitro (1981) L. Mastroianni, Jr. and J. D. Biggers, ed., Plenum Press, New York, N.Y., page 323), the contents of which are hereby incorporated by reference.

Briefly, blastocyts may be transferred non-surgically or surgically into the uterus of a synchronized recipient. Other medium may also be employed using techniques and media well-known to those of ordinary skill in the art. In one procedure, cloned embryos are washed three times with fresh KSOM and cultured in KSOM with 0.1% BSA for 4 days and subsequently with 1% BSA for an additional 3 days, under 5% CO₂, 5% 0₂ and 90% N₂ at 39° C. Embryo development is examined and graded by standard procedures known in the art. Cleavage rates are recorded on day 2 and cleaved embryos are cultured further for 7 days. On day seven, blastocyst development is recorded and one or two embryos, pending availability of embryos and/or animals, is transferred non-surgically into the uterus of each synchronized foster mother.

Foster mothers preferably are examined for pregnancy by rectal palpation or ultrasonography periodically, such as on days 40, 60, 90 and 120 of gestation. Careful observations and continuous ultrasound monitoring (monthly) preferably is made throughout pregnancy to evaluate embryonic loss at various stages of gestation. Any aborted fetuses should be harvested, if possible, for DNA typing to confirm clone status as well as routine pathological examinations.

The reconstituted cell, activated reconstituted cell or embryo, fetus and animal produced during the steps of such method, and cells, nuclei, and other cellular components which may be harvested therefrom, are also asserted as embodiments of the present invention.

The present invention can also be used to produce embryos, fetuses or offspring which can be used, for example, in cell, tissue and organ transplantation. By taking a fetal or adult cell from an animal and using it in the cloning procedure a variety of cells, tissues and possibly organs can be obtained from cloned fetuses as they develop through organogenesis. Cells, tissues, and organs can be isolated from cloned offspring as well. This process can provide a source of “materials” for many medical and veterinary therapies including cell and gene therapy. If the cells are transferred back into the animal in which the cells were derived, then immunological rejection is averted. Also, because many cell types can be isolated from these clones, other methodologies such as hematopoietic chimerism can be used to avoid immunological rejection among animals of the same species as well as between species.

Throughout the description and claims of this specification, the word “comprise” and variations of the word, such as “comprising” and comprises”, is not intended to excluded other additives, components, integers or steps.

The discussion of prior art documents, acts, devices and the like is included in this specification solely for the purpose of providing a context for the present invention. It is not suggested or represented that any or all of these matters formed part of the prior art base or were common general knowledge in the field relevant to the present invention as it existed in Australia before the filing date of this application.

The invention will now be further described by way of reference only to the following non-limiting examples. It should be understood, however, that the examples following are illustrative only, and should not be taken in any way as a restriction on the generality of the invention described above. For example, while the majority of the examples relate to bovine oocytes and granulosa cells, it is to be understood that the invention can also be applied to other animal oocytes as disclosed herein, including for example, sheep, goats and horses.

### EXAMPLE 1

### Methods of Nuclear Transfer Using Granulosa Cells as Donors

Except where otherwise indicated all chemicals were obtained form Sigma Chemical Co. (St Louis, Mo., USA).

In vitro maturation of bovine oocytes (a total of 150 per day) was performed as described in detail earlier (Vajta et al., 1996) with minor modifications. Briefly, oocytes were aspirated from abattoir-derived ovaries, matured in 4-well dishes (Nunc, Roskilde, Denmark) for 24 h in bicarbonate buffered TCM-199 medium (Gibco BRL, Paisley, UK) supplemented with 15% cow serum, 10 IU/ml pregnant mare serum gonadotropin and 51 U/ml human chorionic gonadotropin (Suigonan® Vet, Intervet, Australia) and were incubated under mineral oil at 39° C. in 5% CO₂ in humidified air.

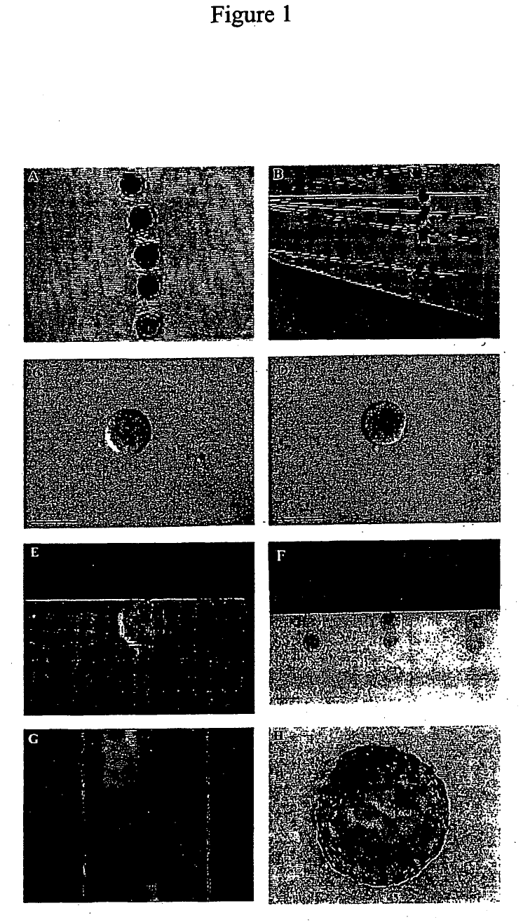

At 19 h after the start of maturation cumulus cells were removed by vortexing. From this point (except where otherwise indicated) all manipulations were performed on a heated stage adjusted to 39° C. Mature oocytes (approximately 110) were selected according to the presence of the first polar body, placed for 5 min into 0.5% pronase (Sigma protease) solution to remove the zona pellucida from the cells. Zona pellucida-free oocytes (approximately 50 to 60, half of the total) were lined up in a 35 mm Petri dish (Falcon, Becton Dickinson Labware, Franklin Lakes, N.J., USA) filled with 4 ml of Hepes buffered TCM-199 medium (TCMH) supplemented with 20% FCS and 10 μg/ml cytochalasin B (refer to FIG. 1A).

Bisection was performed manually under stereomicroscopic control with Ultra sharp Splitting Blades (AB Technology, Pullman, Wash., USA) (refer to FIG. 1B). Bisected oocytes (demi-oocytes) were then collected by swirling in the middle of the dish, placed into the same medium without cytochalasin and the procedure was repeated with the rest of the oocytes.

After completion of the bisection, all demi-oocytes were stained with the fluorochrome Hoechst 33342 dissolved in TCMH and 30% FCS, then placed into 3 μl drops of the same medium formed on the bottom of a 60 mm Falcon petri dish and covered with oil (3 half-oocyte per drop, a total of approx. 70 drop). Following examination under inverted microscopy and ultraviolet illumination, demi-oocytes without chromatin staining (cytoplasts) were selected, collected under a stereomicroscope (a total of approximately 100 to 120) in one well of the original maturation dish under conditions described for maturation and incubated until fusion.

Somatic cells were prepared from granulosa cell monolayers formed in 4-well dishes used 7 to 10 days earlier for maturation. After 5 min incubation in 100 μl of 0.05% trypsin, the well was filled with 800 μl of TCMH and 20% FCS, cells were separated by vigorous pipetting and stored in 1.5 ml Eppendorf tubes at 4° C. until fusion.

Fusions were performed at 21-22 h after the start of maturation. For the first fusion, 15 enucleated oocytes were transferred into TCMH with 2% FCS. 5 μl of the granulosa cell suspension was sedimented to the bottom of the middle section of a 4-well dish filled with TCMH without serum supplementation. Using a finely drawn mouth glass pipette enucleated oocytes were individually exposed for 3 s to 200 μg/ml of PHA (ICN Pharmaceuticals, Australia), then quickly dropped over a single granulosa cell settled to the bottom of the dish (refer to FIGS. 1C and 1D). Following attachment the enucleated oocyte-granulosa cell pair was picked up again, and transferred to a fusion chamber covered with 4 ml of 26-27° C. fusion medium (0.3M mannitol, 0.1 mM MgSO₄, 0.05 mM CaCl₂). The fusion chamber contained parallel platinum wires with a diameter of 1 mm and a separation of 0.8 mm. Using an alternating current (AC) of 15V and 700 KHz (Genaust Electrofusion Machine, Australia). The pair was attached to one wire (somatic cell furthest from the wire—refer to FIG. 1E) then fused with a double DC pulse of 85V, each for 20 μs, 01 s apart. The pair was then carefully removed and incubated in TCM-199 and 20% FCS for 15 to 30 min, when fusion was evaluated. After having fused all 15 enucleated oocytes with granulosa cells, fusion medium was exchanged, and new enucleated oocytes and granulosa cells were prepared for new series of first fusion.

For the second fusion, unfused enucleated oocytes and fused pairs (reconstituted cells) (5 to 10 of each) were first incubated in the fusion medium for 1 to 2 min, then aligned in pair using the same AC pulse, unfused enucleated oocytes attaching the wire (refer to FIG. 1F). A double fusion pulse with the same parameters, but with 45V DC was applied, then the double enucleated oocyte and granulosa cell triplets were incubated in TCMH and 20% FCS for 20 min. Fused reconstituted cells (a total of approximately 40 to 45) were then transferred into a well of a maturation dish and incubated further under conditions described above (Vajta et al., 2002).

Activation was initiated 24 to 26 h after the start of maturation (approx. 3 h after the fusion). Reconstituted cells were first incubated in TCMH containing 10 μM calcium ionophore A23187 for 5 min in air, then in 2 mM 6-dimethylaminopurine (6-DMAP) dissolve in bicarbonate-buffered TCM-199 supplemented with 10% FCS in 5% CO₂ in air for 5 hours.

Embryos were then repeatedly washed 400 μl of SOFaaci medium (Holm et al., 1999) supplemented with 5% cow serum and covered with mineral oil, then randomly distributed into three groups, each of the in the held in the same medium. The first group was individually cultured in 1 μl drops covered with mineral oil. Embryos of the second group were placed in well of the wells (WOWs; Vajta et al., 2000). The third group was individually loaded into 2 μl Drummond microcapillaries (Thouas et al., 2001). All cultures were performed at 39° C., in 5% CO₂ and 90% N₂ (in humidified air).

Two and 7 days after reconstruction, cleavage and blastocyst per embryo rates were evaluated, respectively, under a stereomicroscope. FIG. 1(G) shows a blastocyst developed in the GO system 7 days after fusion. FIG. 1(H) shows the same blastocyst after removal from the GO system. Some of the blastocysts were fixed for future immunohistochemical and ultrastructural investigations; others were vitrified for future embryo transfer experiments.

Statistical analysis of cleavage and blastocyst rates was performed using Pearson Chi-square method were P>0.05 was regarded as significant.

### EXAMPLE 2

### Results of Nuclear Transfer Using Granulosa Cells as Donors

The average efficiency of the main steps and the approximate time required in 7 replicate nuclear transfer experiments using a total of 1016 immature oocytes are summarised in Table 1.

TABLE 1
AVERAGE EFFICIENCY AND APPROXIMATE REQUIRED TIME
FOR STEPS OF ZONA-FREE SOMATIC CELL NUCLEAR
TRANSFER

| | Individual | Cumulative | Time |
|---|---|---|---|
| Procedure | Efficiency | Efficiency | Required |
| PB rate | — | — | 30 min |
| determination | | | |
| Zona removal | 99% | 99% | 10 min |
| Bisection | 89% | 88% | 20 min |
| UV investigation | 91% | 80% | 30 min |
| First fusion | 94% | 75% | 40 min |
| Second fusion | 91% | 69% | 15 min |
| (Related work) | — | — | 35 min |
| Total | | | 180 min |

Oocytes without a well visible polar body (28% of the total) were discarded. However, this loss cannot be attributed to the nuclear transfer method itself; therefore these were not included in the calculation of efficiency. The losses during bisection are the result of lysis observed 5 min after the completion of the procedure. The final accuracy of enucleated oocyte selection was close to 100%. However, a 9% difference mostly attributed to technical failures occurred between the calculated an obtained number. Losses during fusions were almost entirely results of lysis or technical failures, unsuccessful fusions (separated pairs 15-20 min after fusion) were exceptional and usually eliminated with a repeated fusion step.

On average, 105 matured oocytes were used and 35 enucleated oocyte-enucleated oocyte-somatic cell triplets were produced per experiment, and the required time for the work did not exceed 3 hours.

Embryo development rates achieved in the three culture systems are summarised in Table 2. All values were significantly different except for cleavage rates (2-cells or more) for embryos cultured in microdrops versus the WOW system.

TABLE 2
CLEAVAGE AND BLASTOCYST RATES ACHIEVED IN
DIFFERENT CULTURE GROUPS

| Culture system | Cleavage rate | Blastocyst rate |
|---|---|---|
| Microdrop | 25/41 (61%)^{a} | 0/41 (0%)^{a} |
| WOW system | 76/103 (74%)^{a} | 19/103 (18%)^{b} |
| Go system | 47/53 (89%)^{b} | 10/53 (36%)^{c} |

^{a,b,c}Values with different superscripts in the same column mean significant difference.

Generally, embryos cultured in microdrops did not develop beyond the 8- to 16-cell stage; compaction occurred only in the WOW or GO system. Blastocyst formation usually started 6 days after the reconstruction and was completed on Day 7.

The efficiency of the preferred double fusion nuclear transfer method described in the Examples is possibly because the two cells being fused are close in size (in our experiments the cytoplast volume is only half of that of the original oocyte), and the PHA adherence may establish strong membrane contacts on a relatively large area. In contrast to the single-step (enucleated oocyte+enucleated oocyte+blastomere) fusion method of Peura et al. (1998), the double fusion method (first enucleated oocyte+somatic cell to produce a reconstituted cell, then fusing a second enucleated oocyte with the reconstituted cell), describe in the Examples is more convenient and efficient. The use of two enucleated oocytes for reconstruction also means that the cytoplasm volume loss, which is an unavoidable part of the conventional nuclear transfer, can entirely be compensated.

The results of the preferred methods described in the Examples suggest that the methods have considerable potential for the development of cell cloning techniques that meet the requirements for automation of nuclear transfer for the large-scale application of these technologies in agriculture. In particular, the simplified method of somatic cell nuclear transfer greatly reduces reliance on the expensive and technically difficult micromanipulation methods presently used. Furthermore, these improvements have significantly reduced the overall time to conduct successful nuclear transfer.

### EXAMPLE 3

### Simplified Zona-Free Somatic Cell Cloning Techniques

The protocols used in this Example were the same as those described in Example 1, with the following exception.

Reconstituted nuclear transfer embryos were either cultured singly, or as aggregates of 2 reconstituted nuclear transfer embryos and culture was performed either in glass capillaries or in the WOWs, in 4 well Nunclone dishes. After activation the embryos were drawn into the glass tubes or placed into the WOW depressions, either individually, or alternatively, 2 “reconstituted” nuclear transfer embryos were cultured in each glass capillary, or in each WOW depression, and cultured as aggregates after activation.

Tables 3, 4 and 5 show the blastocyst development rates and pregnancy rates from the culture and a transfer of nuclear transfer embryos produced using the techniques described in Example 1. The reconstituted nuclear transfer embryos were cultured either individually (Table 3) or as aggregates of two reconstituted nuclear transfer embryos (Tables 4 and 5).

All experiments reported in Table 3 were performed using one week old granulosa cells except for the last one, where foetal fibroblasts were used. Blastocyst rates were from nuclear transfer embryos cultured singly (i.e. not as aggregates).

Table 4 shows the blastocyst rates achieved from simplified, zona-free nuclear transfer techniques using transgenic donor cells (transfected with bovine a S1 casein gene). Embryos were cultured as aggregates of 2 single reconstituted nuclear transfer embryos. Cultured in either GO or WOW system. Data are based per constructed embryo subjected to activation. The losses as the consequence of fusion and activation have been negligible. 20 to 30 blastocysts can be produced in 3.5 hours (plus activation).

Table 5 shows the pregnancy rates from the transfer of aggregated nuclear transfer embryos from simplified, zona pellucida-free nuclear transfer techniques using transgenic donor cells (fibroblasts transfected with bovine α S1 casein gene). Reconstituted nuclear transfer embryos were either cultured singly, or as aggregates of 2 and culture was performed either in glass capillaries (GO) or in WOWs in 4 well Nun dishes (Lewis et al., 2002).

TABLE 3
BLASTOCYST RATES ACHIEVED 20001/2002

| | | No. of single | | |
|---|---|---|---|---|
| Date of | | reconstituted NT | No | |
| Experiment | Cell Type | embryos into culture | blastocyst | % blastocyst |
| 05.12.01 | granulosa | 44 | 21 | 48% |
| 12.12.01 | granulosa | 40 | 22 | 55% |
| 13.12.01 | granulosa | 59 | 38 | 64% |
| 14.12.01 | granulosa | 53 | 24 | 45% |
| 15.12.01 | granulosa | 54 | 23 | 43% |
| 25.01.02 | granulosa | 39 | 18 | 47% |
| 26.01.02(IL) | granulosa | 21 | 9 | 43% |
| 30.01.02(IL) | granulosa | 19 | 9 | 47% |
| 05.02.02 | fetal | 56 | 25 | 45% |
| | fibroblast | | | |
| TOTAL | | 385 | 189 | 49% |

TABLE 4

| No. of single | | | |
|---|---|---|---|
| reconstituted NT | | | |
| embryos into culture | No. of aggregates | No. blastocyst | % blastocyst |
| 180 | 90 | 35 | 39% per |
| | | | aggregate |
| | | | or 19% per |
| | | | single |
| | | | reconstituted |
| | | | NT embryo |

TABLE 5
PREGNANCY RATES FROM SIMPLIFIED, ZONA-FREE NUCLEAR
TRANSFER TECHNIQUES USING TRANSGENIC DONOR CELLS
(TRANSFECTED WITH BOVINE α S1 CASEIN GENE).

| | No. of recipients | No. of recipients | No. pregnancies |
|---|---|---|---|
| Fresh or | receiving embryos | pregnant at 30-40 | ongoing over |
| vitrified | (no. of embryos) | days (%) | 7 months |
| fresh | 6 (19) | 2 (33%) | 1 |
| vitrified | 5 (16) | 2 (40%) | 1 |
| TOTAL | 11 (35) | 4 (36%) | 2* |

*1 pregnancy lost at 7.5 months (foetus not recovered).
*1 pregnancy lost at 8 months and 1 week gestation. Dystocia - foetus dead after veterinary assisted birth. Calf birth weight 35 kg. Post-mortem at VIAS, Attwood. No significant gross abnormalities detected at P-M. On histopathology, no significant lesions were observed in the brain, thymus, lung, heart, liver, kidney, skeletal muscle or placenta (see VIAS report 01-005483-MW)

### EXAMPLE 4

### Improved Implantation Following the Transfer of Nuclear Transfer Embryos into Recipient Cows

The protocols used were as described in Example 1, with the following exceptions.

The reconstituted single cell nuclear transfer embryos were cultured individually in the WOWs for 4 days (when they were typically between 30 and 60 cells) and then 2 such embryos were combined in single wells for further culture as “aggregates”. Culturing nuclear transfer embryos as such aggregates, increases the cell numbers in the final embryos for transfer and increases pregnancy rates (Peura et al, 1998).

Six 7 day blastocysts were transferred into 6 recipient cows. That is, only 1 embryo was transferred per recipient. Five of the 6 recipient cows were diagnosed pregnant (by ultrasonography) at around day 30 of gestation. In contrast, in Examples 3 and 5 multiple embryos were transferred per recipient which increases pregnancy rates.

### EXAMPLE 5

### Optimisation of Fusion Parameters and Conditions

In this example, research for optimising the fusion parameters and conditions was undertaken. Comparisons were made between fusing somatic cells with 2 or 3 cytoplasts. After activation, reconstituted nuclear transfer embryos were cultured as single embryos, or as aggregates of 2 embryos.

The protocols used in this Example were the same as those used in Example 1, with the following exceptions:

1. A single step fusion was used to fuse the somatic cell and the 2 cytoplasts, instead of the 2 fusion steps used to achieve this end in Example 1.

Somatic cell and cytoplasts (either 2 or 3 cytoplasts per somatic cell) were fused simultaneously. Fibroblasts were used. Required parameters for fusion were calculated according to fibroblast size (Teissie et al., 1998). Either 2 V on the fibroblast surface (112 V=2.22 kV) for 6 μSec (Genaust Electrofusion Machine, Australia) or 3 V on the fibroblast cell surface (166.8 V=0.3 kV) for 4 μSec (BTX Electro Cell Manipulator 200, USA) employing fusion chamber with 0.5 mm gap. The induced potential difference being the steady state value in fibroblasts but will be a minute fraction of it with the larger cytoplast therefore preserving the viability of cytoplast. All fusions were performed using a single pulse.

One half cytoplast was exposed to PHA and attached to the one fibroblast cell by manipulation using finely drawn pipette. Fibroblast/cytoplast pairs together with another half cytoplast (when triplets were made) were equilibrated in the electrofusion medium.

2. Either 2 or 3 cytoplasts were fused with the somatic cell to form the reconstituted nuclear transfer embryo.

3. After activation, nuclear transfer embryos were cultured either as single embryos or as aggregates of 2 reconstituted nuclear transfer embryos.

Seven pregnancies resulted from the transfer of a total of 60 embryos into 16 recipients receiving (i.e. an average of 3.8 embryos were transferred per recipient) At the time of writing there are 3 ongoing pregnancies.

Table 6 compares the fusing 2 or 3 cytoplasts with a somatic cell. Single embryos were cultured in glass capillaries (GO system). Aggregated embryos were cultured in WOWs in 4 well Nunc dishes (2 reconstituted nuclear transfer embryos per WOW)

TABLE 6

| | Single embryo in | | Aggregates (2) | |
|---|---|---|---|---|
| | glass capillary | | embryos per WOW | |
| | 2-cytoplast | 3-cytoplast | 2-cytoplast | 3-cytoplast* |
| Blastocyst | 10.5% | 23.3% | 14.9% | 63.6% |
| Rates | (24/228) | (7/30) | (30/189) | (35/55) |

*Fusions were Performed with BTX machine with predicted values (166 V for 4 μSec) while others were performed with GA machine (112 V for 6 μSec.). Reconstituted

Table 7 shows the number of transferred embryos and pregnancy rates

TABLE 7
Number of embryos transferred (derived from either single or
aggregated reconstituted nuclear transfer embryos)

| | No. of | No. of | Pregnancy | | Ongoing |
|---|---|---|---|---|---|
| | Embryos | Recipients | rate | Lost | Pregnancies |
| Fresh | 7 | 2 | 50.0% (1/2) | 1 | 0 |
| Cryo- | 53 | 14 | 42.9% (6/14) | 3 | 3 |
| preserved | | | | | |
| Total | 60 | 16 | 43.8% (7/16) | 4 | 3 |

It was noted that less time was required time for the process. In particular, the number of steps (re-checking and re-fusion of SC-cytoplast complex, 2^{nd} fusion with an another oocyte) have been cut down with optimised fusion parameters and conditions without sacrificing the efficiency, Fusion process can be completed in a very short time, however enucleation process still takes almost 60-70% of the time spent on the process. This will be the major target for automatisation of the system.

Predicted voltages and pulse duration are efficient for simultaneous fusion. Predicted values (calculated according to 9 μm fibroblast) were tested with BTX machine for simultaneous fusion of somatic cell+cytoplasts, fusion and blastocysts rates were determined.

Oocytes were not selected according to PB or morphological appearances since September in order to speed up to system. The same no selection criteria was applied to the transferred embryos.

PVA was added to fusion medium to prevent stickiness and membrane rupture of oocytes therefore lyses. Although it is suggested that fusion medium should be prepared every 2 weeks, medium was frozen in aliquots and no harmful effect was noted. Demo-oocytes were incubated in the separate well containing fusion medium before introducing into the fusion chamber.

Existing fusion protocol has been simplified and improved by introduction of a single step fusion. Somatic cell and cytoplasts are fused simultaneously (10-12 nuclear transfer at the same time) compared to existing two step protocol (somatic cell+cytoplast and then cytoplast+cytoplast). Required time for existing protocol cut-down considerably.

Also fusion parameters were calculated and tested according to the existing formula (Teissie et al., 1997). Somatic cell and zona pellucida-free, enucleated oocytes were fused simultaneously (10-12 nuclear transfer at the same time). With the new parameters (3 V on the cell surface for 4 μSec, the induced potential difference will be its steady state value in fibroblasts but will be a minute fraction of it with the larger cytoplast and this will preserve the cell viability of this larger partner.

### EXAMPLE 6

### Nuclear Transfer Method Using Murine and Ovine Oocytes Compared to Bovine Oocytes

The protocols shown in Example 1 were used in experiments with ovine and murine oocytes. For example, ovine and murine oocytes were harvested, treated to remove the zona pellucida and enucleated in a similar fashion to the bovine oocytes referred to in Example 1. The somatic cells used were fibroblasts.

Table 8 shows the fusion rates in reconstituted sheep embryos following simultaneous fusion of 2 cytoplasts and a somatic cell (fibroblast) at 55 V (1.1 kV) and 112 V (2.2 kV) for 6 μSec.

TABLE 8
FUSION RATES IN RECONSTITUTED ZONA PELLUCIDA-FREE
OVINE EMBRYOS

Voltage

| | 55 V (1.1 kV) | 112 V (2.2 Kv) |
|---|---|---|
| Fusion Rates | 30.7% (4/13) | 61.5% (4/13) |

Table 9 shows the fusion rates in reconstituted ovine embryos following simultaneous fusion of 2 cytoplast and ovine somatic cell (fibroblast) at 112 V (2.2 kV) for 6 μSec in 2 replicates of experiments. No of chromatins in each embryo was checked 3 hours after fusion under the fluorescent microscope.

TABLE 9
CHROMATIN IN RECONSTITUTED ZONA PELLUCIDA-FREE
OVINE EMBRYOS

| Groups | % (No) |
|---|---|
| Control | 10.0% (1/10) |
| Chromatin | 64.1% (25/39) |
| Single Chromatin | 58.9% (23/35) |
| Double Chromatin | 5.70% (2/35) |
| No Chromatin | 35.9% (14/39) |

Table 10 shows the lyses rates of reconstituted sheep nuclear transfer embryos following simultaneous fusion of 2 cytoplast and somatic cell (fibroblast) at 112 V (2.2 kV) for 6 μSec in 2 replicates of experiments

TABLE 10
LYSIS RATES OF RECONSTITUTED ZONA PELLUCIDA-FREE
OVINE NUCLEAR TRANSFER EMBRYOS

| Fusion Rates | % (no) |
|---|---|
| No oocytes | 100% (138; expected demi oocytes = 276) |
| Demi-oocytes | 83.4% (231) |
| Lyses following splitting | 16.3% (45) |
| No of cultured embryos | 43 |

TABLE 11
LYSIS FOLLOWING BISECTION OF ZONA PELLUCIDA-FREE
MURINE OOCYTES

| Groups | % (No) |
|---|---|
| No oocytes | 100% (36 expected demi oocytes = 72) |
| Demi-oocytes | 12.5% (9) |
| Lyses following splitting | 87.5 (63) |

TABLE 12
BLASTOCYST RATES OF INTERSPECIES
(BOVINE CYTOPLAST + RODENT FIBROBLAST)
RECONSTITUTED EMBRYOS FOLLOWING
SIMULTANEOUS FUSION OF 2 CYTOPLAST AND
SOMATIC CELL (FIBROBLAST) AT V (2.2 KV) FOR 6 μSEC.

| | Fresh mouse | Dried mouse |
|---|---|---|
| | fibroblast | fibroblast |
| No of reconstituted embryos | 19 | 18 |
| Cleavage | 12 (63.2%) | 8 (44.4%) |

### REFERENCES CITED

Colman, A (1999). Somatic cell nuclear transfer in mammals: progress and applications. Cloning 1:185-200.
Galli, C, Duchi, R Moor, R M and Lazzari, G. (1999). Mammalian leucocytes contains information necessary for the development of a new individual. Cloning 1: 161-170.
Holm, P, Booth, P J, Schmidt, M H, Greve, T. and Callesen, H. (1999). High bovine blastocyst development in a static in vitro production system using SOFaa medium supplemented with sodium citrate and myo-inositol with or without serum-proteins. Theriogenology: 52, 683-700.
Lewis, I M, Vajta, G, French, A J, Hall, V J, Korfiatis, N A, Ruddock, N T, Travers, M J, Travers, R L and Trounson, A O. (2002). Pregnancy rates from simplified zona-free somatic cell cloning and traditional zona-enclosed cloning in cattle. Theriogenology 57, 431.
Lewis, I M, Munsie, M J, French, A J, Daniels, R and Trounson, A O. (2001). The cloning cycle: from amphibia to mammals and back. Reproductive Medicine Reviews 9, 13-33.
Peura, T T, Lane, M, Lewis, I M and Trounson, A O. (1998). The effect of recipient oocyte volume on nuclear transfer in cattle. Mol. Reprod. Dev. 50, 185-191.
Teissie, J and Ramos, C. (1998). Correlation between electric field pulse induced long-lived permeabilization and fusogenicity in cell membranes. Biophys. J. 74:1889-98.
Thouas, G A, Jones, G M and Trounson, A O. (2001). A novel method of micro-culture of mouse zygotes to the blastocyst stage—the “GO” culture system. Human Reproduction 16,168.
Trounson, A. (2001). Nuclear transfer in human medicine and animal breeding. Reprod. Fertil. Dev. 13: 31-9.
Vajta G., Lewis I M, Korfiatis N A, Travers R L, Trounson A O. (2002). Bovine somatic cell cloning without micromanipulators: optimization of certain parameters. Theriogenology 57, 453.
Vajta, G, Holm, P, Greve, T. and Callesen, H. (1996). Factors affecting survival rates of in vitro produced bovine embryos after vitrification and direct in-straw rehydration. Theriogenology, 45: 191-200.
Vajta, G, Peura, T T, Holm, P, Páldi, A, Greve, T and Trounson, A O and Callesen, H. (2000). New method for culture of zona-included or zona-free embryos: the Well of the Well (WOW) system. Mol. Reprod. Dev. 55: 256-254.
Wells, K D and Powell, A M. (2000). Blastomeres from somatic cell nuclear transfer embryos are not allocated randomly in chimeric blastocysts. Cloning, 2: 9-22.

What is claimed is:

1. A method of nuclear transfer comprising transferring a somatic cell or cells, or a somatic cell nucleus or nuclei, into two or more zona pellucida-free, enucleated non-human mammalian oocytes to increase the oocyte cytoplasmic volume compared to nuclear transfer to one such oocyte.

2. The method of claim 1, wherein the somatic cell or somatic cell nucleus is attached to said two or more zona pellucida-free, enucleated oocyte, prior to the transferring.

3. The method of claim 1, wherein the oocytes are isolated from oviducts and/or ovaries of said non-human mammal.

4. The method of claim 3, wherein the oocytes are isolated by aspiration.

5. The method of claim 1, wherein the somatic cell, nucleus or nuclei and/or the oocytes are isolated from an ungulate mammal of the bovid or equid family.

6. The method of claim 5, wherein the bovid mammal is male or female bovine, sheep or big-horn sheep, and the equid mammal is a horse, pony, donkey or mule.

7. The method of claim 6, wherein the bovine mammal is a member of the species Bos taurus, Bos indicus or Bos buffaloes.

8. The method of claim 1, wherein the oocytes are freed of the zona pellucida by physical manipulation, chemical treatment or enzymatic digestion.

9. The method of claim 1, wherein the oocytes are enucleated by aspiration, physical removal, use of a DNA-specific fluorochrome, or ultraviolet irradiation.

10. The method of claim 1, wherein the somatic cell is an epithelial cell, a neural cell, an epidermal cell, a keratinocyte, a hematopoietic cell, a melanocyte, a chondrocyte, a lymphocyte, an erythrocyte, a macrophage, a monocyte, a fibroblast, a cardiac muscle cells, or another muscle cells.

11. The method of claim 10, wherein the somatic cell is a transgenic cell.

12. The method of claim 1, wherein the somatic cell or nucleus is transferred into the oocyte by fusion.

13. The method of claim 12, wherein the fusion is promoted by a fusion-promoting agent selected from the group consisting of polyethylene glycol, trypsin, dimethylsulfoxide, a lectin, an agglutinin, and a virus.

14. The method of claim 12, wherein the fusion is achieved by electrofusion wherein one or more electrical pulses is delivered to the two or more oocytes and the somatic cell or nucleus.

15. A non-human mammal obtained by a method that comprises the method of claim 1.

16. A method of producing a non human mammalian embryo of 8-128 cells from a reconstituted cell which reconstituted cell is an embryo, comprising:
(i) inserting a desired somatic cell or somatic cell nucleus of a non-human mammal into two or more zona pellucida-free, enucleated oocytes, under conditions suitable for the formation of a reconstituted cell which is an embryo;
(ii) activating the reconstituted cell; and
(iii) culturing said reconstituted cells until one or more 8- to 128-cell embryos develop.

17. The method of claim 16, wherein step (iii) comprises co-culturing two or three of said reconstituted cells.

18. The method of claim 16, wherein the reconstituted cells are cultured as two or more cells in step (iii) until said embryos of between 8 and 128 cells develop, at which time two or more embryos are combined and co-cultured as aggregates.

19. the method of claim 16 wherein, in step (i), the somatic cell or nucleus is attached to said two or more oocytes prior to, or contemporaneously with, said inserting.

20. A method for cloning a non-human mammal comprising the steps of:
(i) inserting a desired somatic cell or somatic cell nucleus from a non-human donor mammal into two or more zona pellucida-free, enucleated oocytes, under conditions suitable for the formation of reconstituted oocytes;
(ii) activating the reconstituted oocytes to develop into an embryo;
(iii) culturing the embryo beyond a two-cell developmental stage; and
(iv) transferring the cultured embryo into a female non-human mammalian host such that the embryo develops into a fetus in the host.

21. The method of claim 20, wherein the somatic cell, the somatic cell nucleus and/or the oocytes are from an ungulate mammal that is a wild or domestic bovid or equid.

22. The method of claim 21, wherein the ungulate mammal is a male or female bovine, sheep, horse, pony, donkey, or mule.

23. The method of claim 22, wherein the bovine mammal is a member of the species Bos taurus, Bos indicus or Bos buffaloes.

24. A non-human mammal obtained by the method of claim 20.

25. A cell, tissue or organ obtained from the non-human mammal of claim 24.

26. The method of claim 20, wherein the inserting of step (i) is by fusion between said somatic cell or nucleus and said oocytes.

27. The method of claim 26, wherein the fusion is accomplished by electrofusion is induced by delivery of one or more electrical pulses to the oocytes and the somatic cell or somatic cell nucleus.

28. The method of claim 20, wherein said two or more oocytes of step (i) are fused prior to said activating step (ii), to increase the cytoplasmic volume.

29. The method of claim 20 wherein, in step (i) the somatic cell or nucleus is attached to said two or more oocytes prior to said inserting step.

30. The method of claim 29, wherein the attaching comprises exposing said two or more oocytes and said somatic cell or nucleus to a lectin or agglutinin that causes cells to agglutinate or adhere to one another.

31. The method of claim 20, wherein the activating is by (i) electric pulse, (ii) chemical shock, (iii) penetration by sperm, (iv) increasing intracellular levels of divalent cations or (iv) reducing phosphorylation.

32. The method of claim 20, wherein, in step (iv), the embryo is transferred into the uterus of a synchronized recipient.

33. The method of claim 20, wherein the cloned mammal is transgenic or genetically engineered, and the method further comprises, prior to step (i); the step of altering the somatic cell or nucleus by inserting, deleting or modifying a desired gene or genes.

34. The method of claim 20, wherein the somatic cell is an epithelial cell, neural cell, epidermal cell, keratinocyte, hematopoietic cell, melanocyte, chondrocyte, lymphocyte, erythrocyte, macrophage, monocyte, fibroblast, cardiac muscle cell, or other muscle cell.

35. The method of claim 20, wherein the somatic cell is a transgenic cell modified by insertion, deletion or modification of a desired gene or genes.
